(19) United States
(12) Patent Application Publication
(10) Pub. No.: US 2006/0293216 A1
(43) Pub. Date: Dec. 28, 2006

(54) Pharmaceutical compositions comprising an active agent and chitosan for sustained drug release or mucoadhesion

(75) Inventors: Jo Klaveness, Trondheim (NO); Etnar Mustaparta, Trondheim (NO); Bjarne Brudeli, Trondheim (NO); Olaw Smidsrod, Trondheim (NO); Kjell Morten Varum, Trondheim (NO)

Correspondence Address: SUGHRUE MION, PLLC, 2100 PENNSYLVANIA AVENUE, N.W., SUITE 800, WASHINGTON, DC, 20037, US

(73) Assignee: ADVANCED BIOPOLYMERS AS, Trondheim (NO)

(21) Appl. No.: 10/544,313

(22) PCT Filed: Feb. 6, 2004
(86) PCT No.: PCT/GB04/00477

§ 371 Date: May 16, 2006

(30) Foreign Application Priority Data
Feb. 6, 2003 (GB) 0302738.0

(51) Int. Cl. A61K 38/17; A61K 31/722; A61K 31/60; A61K 31/192

(52) U.S. Cl. 514/2; 514/165; 514/55; 514/569; 514/570

(57) ABSTRACT

The invention provides a pharmaceutical composition comprising a physiologically active agent and a release sustaining or mucoadhesive agent, characterized in that said release sustaining or mucoadhesive agent comprises a chitosan having a F_{A} of from 0.25 to 0.80.

The invention relates to pharmaceutical compositions containing a physiologically active agent, i.e. a drug, and a release sustaining or mucoadhesive agent which serves to prolong the release of the active agent from the composition or retain the composition in contact with a mucous membrane, in particular compositions wherein the release sustaining or mucoadhesive agent comprises a chitosan.

Chitosan is the product of complete or partial deacetylation of chitin.

Chitin is a natural nitrogenous mucopolysaccharide of formula (C₈H₁₃NO₅)ₙ which occurs in the exoskeletons of invertebrates and also in funghi. In particular it is a major component of the exoskeletons of crustacea such as shrimp, crab, prawn and lobster. More particularly chitin is poly N-acetyl-D-glucosamine. Thus chitin consists of (1→4)-linked 2-acetamido-2-deoxy-β-D-glucose (GlcNac; the A-unit). The physical structure of chitin is highly ordered, and the most abundant form is α-chitin which is available as a waste material from the shellfish food industry. In α-chitin the chains are antiparallel, and extensively hydrogen-bonded. Another form is β-chitin, which can be isolated from, for example the pen of the squid Loligo and the spines of the diatom Thalassiosira fluviatilis. In β-chitin the chains are parallel, and the chains are less hydrogen-bonded compared with α-chitin.

Chitin is insoluble in water, even at acidic pH-values, and in most organic solvents. This has served to limit the applications for which it is used.

The N-acetyl groups in chitin can be cleaved off to yield the product known as chitosan. Chitosan has many known uses, e.g. in pharmaceutical and cosmetic compositions, and as fillers, absorbants, carriers and supports.

Chitosan may be regarded as a family of water-soluble polysaccharides consisting of (1→4)-linked A-units and units of 2-amino-2-deoxy-β-D-glucose (GlcN; the D-unit) in varying relative abundances and sequences.

The distinction here between chitin and chitosan is based on the insolubility of chitin in dilute acid solution and the solubility of chitosan in the same dilute acid solution (see Roberts, G. A. F., “Chitin Chemistry” (1991), pages 6-7).

The definition of fully water-soluble chitosan given on page 6 of Roberts (supra) is related to the fact that chitosans are generally only soluble in water when the free amino groups of D-units are protonated. Such protonation can be achieved by the addition of a controlled amount of an acid, e.g. acetic acid. However, chitosan can also be prepared in different salt forms, i.e. with a protonated amino-group in the D-units and a negatively charged counterion (e.g. formate, acetate, chloride or another negative ion), which make it soluble in water without the addition of an acid. Procedures for the preparation of such chitosan salts are described in the literature (see for example Draget et al, Biomaterials 13:635-638 (1992), V\dot{a}rum et al. Carbohydrate Polymers 28:187-193 (1995), and U.S. Pat. No. 5,599,916).

One parameter used to characterize chitosans is F_{A}, the relative-fraction of the saccharide units which are A rather than D units.

To illustrate the structure of chitosan, the following schematic representation of the chemical structure of three different chitosans with varying compositions of A and D-units are given:

DDDDDDDDDDDDDDDDDDDDDDDDDDDDDDDDDDDDDDDD

Part of a fully N-deacetylated chitosan molecule

(F_{A}=0.00)

DDDADDADDDDDAADDADDDDDADADDDDAADDDDADDDD

Part of a partially N-acetylated chitosan molecule

(F_{A}=0.25)

DAAADDADDDDAAAADADDADDADDDDADAAAADDAADAA

Part of a partially N-acetylated chitosan molecule

(F_{A}=0.50)

The presence of one monomer residue with a hydrophilic and protonizable amino group and another monomer residue with a hydrophobic acetyl group, where the relative amounts of the two monomers can be varied, can affect chitosan's physical properties in solution and in the gel and solid states, as well as its interactions with other molecules, cells and other biological and non-biological matter. However, the commercial use of chitosan has so far been limited to chitosan samples with a low fraction of acetylated units (F_{A}<0.15) due partly to the lack of inexpensive methods to prepare other chitosans on a large scale, and due partly to the limited scientific understanding of the functional properties of chitosans with a higher F_{A}.

It should be noted that besides deacetylation, in the production of chitosan from chitin, depolymerisation may also occur and chitosan can be produced with a wide range of degrees of acetylation and a wide range of molecular weights. In general, however, one remaining problem with commercially available chitosan is its insolubility at physiological pH values.

The production of chitosan from chitin is generally carried out as either a homogeneous reaction or as a heterogeneous reaction. In the homogeneous reaction chitin is suspended in alkali and the suspension is cooled with ice to bring the chitin into solution; in the heterogeneous reaction particulate chitin is dispersed in a hot alkaline solution, generally sodium hydroxide. In the case of the homogeneous reaction, the F_{A} of the chitosan obtained is generally 0.3 to 0.7. In the case of the heterogeneous reaction, the F_{A} of the chitosan obtained is generally in the range of 0 to 0.15. Where a chitosan with a different degree of deacetylation is required it may be necessary to re-acetylate the chitosan. In the case of the homogeneous reaction, the remaining N-acetyl groups are generally randomly located along the polymeric backbone of the chitosan product. In the case of the heterogeneous reaction, a small fraction of insoluble chitin-like material is most often present in the product together with an acid-soluble fraction with a near random distribution of acetyl groups along the polymeric backbones.

Descriptions of prior art deacetylation procedures may be found in: U.S. Pat. No. 4,195,175; V\dot{a}rum et al, pages 127-136 in “Advances in chitin chemistry”, Ed. C. J. Brine, 1992; Ottøy et al, Carbohydrate Polymers 29:17-24 (1996); Sannan et al, Macromol. Chem. 176:1191-1195 (1975); Sannan et al, Macromol. Chem. 177:3589-3600 (1976); Kurita et al, Chemistry Letters 1597-1598 (1989); and CA-A-2101079.

Enhanced performance, in several applications, has recently been found for more highly acetylated chitosan fractions (see Smidsrøod et al, pages 1 to 11, in “Chitin and Chitosan—Chitin and Chitosan in Life Science”; Eds. T. Uragami et al., Kodansha Scientific, Japan (2001) (ISDN 4-906464-13-0)). Of importance is increased solubility at neutral pH-values, a controllable degradation rate by lysozymes, strong interactions with hydrophobic surfaces (e.g. fat particles and cell surfaces) thereby giving enhanced fat binding properties and flocculation, enhanced destabilisation effects on oil-in-water-emulsions, and extended utility in a number of cosmetic, nutraceutical and biomedical applications.

More highly acetylated chitosans have also recently been shown to flocculate bacterial cells more effectively (see Strand et al. Biomacromolecules 2:126-133 (2001)).

However the known procedures for preparation of more highly acetylated chitosans suffer from disadvantages which make them unsuitable for upscaling to industrial production.

Thus, for example, for the heterogeneous deacetylation process without swelling, it is necessary to extract the product with an acid in order to separate the unreacted chitin from the water-soluble chitosan; this involves removal of water in addition to reduced yield of the highly acetylated chitosan product.

The reacetylation of a highly deacetylated chitosan, in addition to the deacetylation step, involves solubilization of the chitosan, use of organic chemicals such as acetic anhydride and methanol, and isolation of the final product.

The homogeneous deacetylation procedure involves solubilisation of the chitin by addition of ice, and isolation of the chitosan from the solution. Moreover, to avoid the chitin solution having too high a viscosity, large volumes of aqueous lye are needed in the reaction medium. This homogeneous deacetylation procedure therefore results in a more expensive product compared to the product of a heterogeneous deacetylation procedure.

Advanced Biopolymers AS have recently found that if in the heterogeneous deacetylation reaction the chitin is first subjected to a prolonged low temperature alkaline swelling stage a chitosan product may be obtained with a more random distribution of residual N-acetyl groups along the polymeric chains, with a degree of deacetylation which can be as low or high as desired, with a degree of depolymerisation which may if desired be lower than in the conventional products, and if desired with an enhanced water-solubility at physiological pHs. This novel chitosan production process is described in WO 03/011912 the contents of which are incorporated herein by reference.

Using this new process, chitosans having whatever F_{A} as desired may be produced and in particular pH neutral water soluble chitosans with relatively high F_{A} values may be produced.

While it has been known that chitosan may be used as a release sustaining agent in pharmaceutical compositions, we have now surprisingly found that the release sustaining effect is dependent on the F_{A} of the chitosan used, with higher F_{A} chitosans serving to prolong the release period. Thus pharmaceutical compositions can be produced with the desired drug release profile by appropriate selection of one or more chitosans with one or more F_{A} values.

We have also found that the chitosans may be used as mucoadhesive agents where they serve not only to maintain a drug composition in contact with a mucous membrane but also to permit sustained release of the drug from the composition.

Thus viewed from one aspect the invention provides a pharmaceutical composition comprising a physiologically active agent and a release sustaining or mucoadhesive agent, characterized in that said release sustaining or mucoadhesive agent comprises a chitosan having an F_{A} of from 0.25 to 0.80, especially 0.30 to 0.60, particularly 0.33 to 0.55.

Viewed from a further aspect the invention provides a pharmaceutical composition comprising a physiologically active agent and a release sustaining or mucoadhesive agent, characterized in that said release sustaining or mucoadhesive agent comprises at least two chitosans having different F_{A} values, at least one said chitosan preferably having an F_{A} value in the range 0.25 to 0.80, especially 0.30 to 0.60, particularly 0.33 to 0.55.

The pharmaceutical compositions of the invention will typically be in forms suitable for administration into the gastrointestinal tract, e.g. orally or rectally. Typical such forms include tablets, coated tablets, capsules, powders, gels, solutions, dispersions, suspensions and syrups. Tablets, capsules and solutions are preferred. Such compositions may also include physiologically tolerable carriers and excipients, e.g. conventional formulation components such as flavours, solvents (especially water), fillers, stabilizers, antioxidants, pH modifiers, viscosity modifiers, sweeteners, colorants, etc. The compositions may be prepared by conventional formulation techniques.

While the most preferred administration route for the compositions of the invention is oral, alternative administration routes are to the nose, eyes and mucous membranes (e.g. vaginal, sublingual, etc). For this purpose, the compositions may typically take the form of powders, sprays, solutions, creams, ointments, pessaries, suspensions, dispersions, films, etc. Typical drugs that may be delivered in this way, in particular nasally, include insulin, hormones, encephalins, vaccines and other peptide drugs.

The compositions of the invention may additionally be formulated such that the chitosan and/or the physiologically active agent is present in a solid or liquid crystalline micro- or nano-structure, e.g. a nanoparticle, a liposome, a micelle, a reversed micelle, or a fragmented cubic or hexagonal phase liquid crystal. The chitosan itself moreover may be used to encapsulate (again in nano- or microparticles) the physiologically active agent. Such uses of chitosan (of whatever F_{A}) are novel and form a further aspect of the invention.

It is especially preferred however that in the compositions of the invention the chitosan and the active agent are mixed at the molecular level. This may be achieved by solvent removal from a solution of the active agent and the chitosan. Compositions containing chitosan and physiologically active agents admixed at the molecular level are new and form a further aspect of the present invention. Viewed from this aspect the invention provides a pharmaceutical composition comprising admixed at the molecular level a solid mixture of a chitosan and a physiologically active agent, e.g. produced by solvent removal from a solution of the active agent and the chitosan. In such compositions the chitosan is preferably but not essentially a chitosan or chitosan mixture in accordance with the other aspects of the invention.

The physiologically active agent in the compositions of the invention may be any desired drug compound or mixture of drug compounds, particularly drug compounds for which a sustained availability for uptake from the gastrointestinal tract is desired. The physiologically active agent is especially preferably a compound with a relatively low molecular weight (e.g. up to 500 g/mol) or a protein or peptide with a molecular weight of up to 7000 g/mol. Particular mention may be made of analgesics, antiinflammatories, hormones, antiparasitics, antineoplastics, antihypertensives, anti-ulcer drugs, and antidepressants. Particular mention may also be made of drugs which affect the peripheral and central nervous systems, drugs which affect renal function, drugs which affect electrolyte metabolism, drugs which affect gastrointestinal function, drugs which are used in chemotherapy of cancers, cardiovascular drugs and drugs which act on the blood and blood-forming tissues. Especially preferably the drug compound is an acidic water-soluble drug, e.g. one such as acetylsalicylic acid and other NSAIDs (such as ibuprofen), antibiotics (for example penicillin) and anticoagulants (for example varfarin). The content of the physiologically active agent in the compositions of the invention will of course be dependant on the nature of the active agent, the severity of the condition to be treated, and the age, sex and bodyweight of the individual being treated. Typically however the content will be within 10% of the content of the same active agent in comparable conventional formulations.

The chitosan used in the compositions of the invention is preferably a fully water-soluble chitosan, particularly a chitosan soluble in water at the pH's encountered in the gastrointestinal tract or at the site of administration if administration is not oral, more particularly a chitosan which is water-soluble at pH's of 3 to 7, especially 5 to 7, more especially 6 to 7.

By “fully water-soluble chitosan” as used herein, is meant a chitosan that can be fully dissolved, that is more than 97% wt dissolved in a dilute acid solution, for example as a 1% w/v solution of the chitosan in 1% w/v acetic acid.

The chitosan used is preferably produced using the processes described in WO 03/011912.

Particularly desirably a combination of chitosans with different F_{A} values is used, e.g. at least two chitosans with F_{A} values differing by at least 0.1, more preferably by at least 0.2, and even more preferably at least three such chitosans. In this embodiment, the chitosans are preferably used in amounts of at least 0.5 parts by weight relative to the most abundant chitosan which can be deemed to be used in an amount of 1 part by weight.

The chitosans used preferably have F_{A} values above 0.25; however where two or more chitosans are used one or more may have F_{A} values below 0.25, e.g. below 0.2, for example 0.05 to 0.19.

The chitosans used according to the invention may have a weight average molecular weight (M_{w}) within a very broad range, e.g. 1000 to 5000000 g/mol. Preferably however M_{w} is 10000 to 3000000 g/mol, especially 20000 to 2000000 g/mol.

The chitosans will be used in quantities sufficient to achieve the desired release sustaining and/or mucoadhesive effect. Typically this may be 5 to 98% wt of the composition, preferably 20 to 90% wt, excluding the weight of any solvent or casing. The weight ratio of chitosan to drug may vary over a wide range depending on factors such as the nature of the drug, the F_{A} and molecular weight of the chitosan, the drug administration form (i.e. tablet, solution, etc) and the desired drug release profile. Especially preferably the chitosan will provide from one glucosamine unit to one chitosan molecule per drug molecule. Generally however the weight ratio of chitosan to drug will be in the range 20:1 to 0.5:1, preferably 10:1 to 1:1, especially 5:1 to 2:1.

The invention will now be illustrated further by reference to the following non-limiting Examples and the accompanying drawings in which:

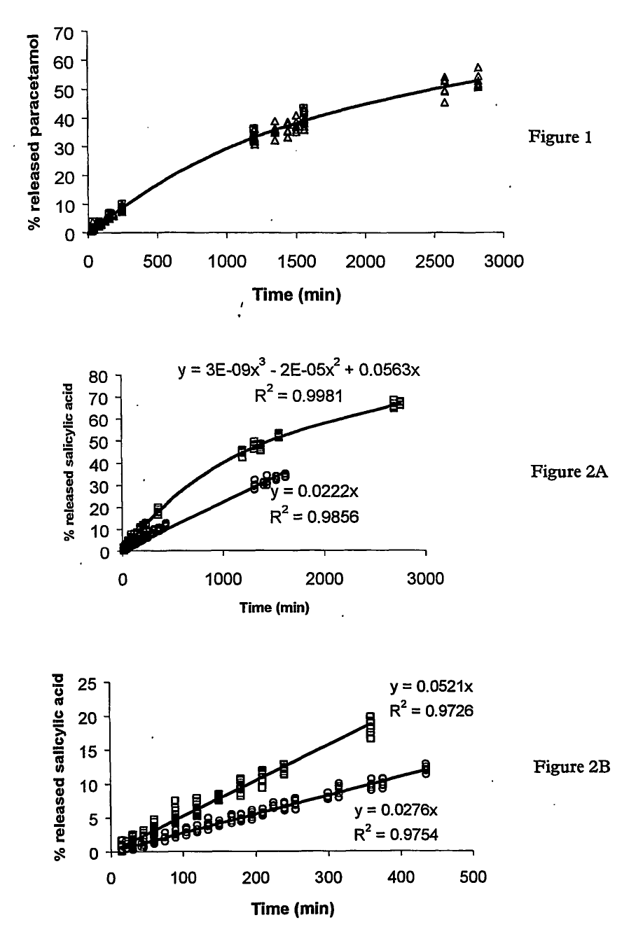

FIG. 1 is a plot of the time course of release of Paracetamol from a solution (10 ml) containing Paracetamol (10 mM in 154 mM NaCl, pH 4.5) without (□) and with (Δ) chitosan (3% (w/v)) to a 1 L reservoir containing 154 mM NaCl, pH 4.5; and

FIGS. 2A and 2B are plots of the time course of release of salicylate from a solution (10 ml) containing salicylate (30 mM in 154 mM NaCl, pH 4.5) without (□) and with (Δ) chitosan (3% (w/v)) to a 1 L reservoir containing 154 mM NaCl, pH 4.5. FIG. 2B shows the initial time course of the release of the drug.

### EXAMPLE 1

Capsules Comprising Acetyl Salicylic Acid

7.5 g acetyl salicylic acid

25 g chitosan F_{A} 0.45*
lactose q.s.

*—Produced as described in WO 03/011912

The components are mixed and filled in hard gelatin capsules. Each capsule contains 75 mg acetyl salicylic acid. The main indication for this drug composition is for anticoagulant prophylaxis.

### EXAMPLE 2

Capsules Comprising Ibuprofen

20 g ibuprofen

17 g chitosan F_{A} 0.36*
lactose q.s.

*—Produced as described in WO 03/011912

The components are mixed and filled in hard gelatin capsules. Each capsule contains 200 mg ibuprofen. This composition is used as an analgesic.

### EXAMPLE 3

Insulin Formulation for Nasal Delivery

10 mL Insulin Ultratard 100 IE/ml (from Novo Nordisk)

300 mg Chitosan glutamate F_{A} 0.46

Chitosan glutamate (F_{A} 0.46) is prepared by conventional methods from chitosan (F_{A} 0.46) (produced as described in WO 03/011912) and glutamic acid. Chitosan glutamate is dissolved in Insulin Ultratard. Insulin Ultratard is a suspension of crystalline insulin. The suspension is filled into a nasal delivery system.

### EXAMPLE 4

Relative Studies

The chitosan used in this Example was prepared from a chitosan produced as described in WO 03/011912 (F_{A} 0.41, [η]=1060 ml/g), which was depolymerized and at the same time converted to the chitosan hydrochloride salt using 3M ethanolic HCl. Excess ethanolic chitosan was removed, the chitosan washed with excess 70% ethanol, 96% ethanol and finally dried to obtain the chitosan hydrochloride salt. The intrinsic viscosity was determined to 200 ml/g, corresponding to a number-average molecular weight of 40 000 (Anthonsen et al., 1993, Carbohydr. Polym. (1993) 22 193-201).

30 mM Salicylic acid was dissolved in distilled water upon addition of equimolar amounts of sodium hydroxide, and sodium chloride was added to a final concentration of 154 mM. The pH was adjusted to 4.5.

10 mM Paracetamol was dissolved in 154 mM NaCl at pH 4.5.

Each of the solutions containing salicylate or paracetamol was added to a small glass vial (10 ml) equipped with a dialysis membrane (d=14.3 mm, cut off 10-12 kDa). The glass vials were placed in a 1 litre reservoir containing 154 mM NaCl, pH 4.5. Samples of 3.0 ml were regularly withdrawn from the reservoir and the absorbance was measured at 297.0 nm (salicylic acid) and 243.3 nm (paracetamol). Each experiment was run with 6 parallels.

The same experiment was performed with paracetamol and the salicylate solutions to which had been added 3 (w/v)% of the chitosan.

Neutral Drug (Paracetamol)

The diffusion of paracetamol through the dialysis membrane was followed for 2 days in the presence and absence of chitosan and the results are shown in FIG. 1 of the accompanying drawings. No difference in the release profile of the neutral drug paracetamol with and without chitosan could be detected.

Negatively Charged Drug (Salicylate)

The diffusion of salicylate through the dialysis membrane was followed in the same way as for paracetamol, and the results are as shown in FIG. 2 of the accompanying drawings. A clear difference between the release of the negatively charged drug with and without chitosan was seen when comparing the data of FIG. 2 with FIG. 1.

### EXAMPLE 5

Release of Acetylsalicylic Acid from Chitosan

Acetylsalicylic acid (100 mg) and chitosan (various degrees of acetylation) (250 mg) were added to a diluted aqueous HCl solution at pH 2 (10 ml). The mixture was stirred for 30 minutes at 80° C., cooled to room temperature, transferred to a dialysis tube (cut off 12-14 kDa) and dialysed against tris buffer pH7 (100 ml). The amount of acetylsalicylic acid in the dialysate was determined by UV.

An experiment without chitosan was performed as a comparison.

The amounts of acetylsalicylic acid in dialysate are shown in Table 1 as a percentage of maximum detected amounts.

TABLE 1

| Time for | | | |
|---|---|---|---|
| dialysis | Chitosan | Chitosan | |
| (hours) | F_{A} = 0.46, [η] = 1230 | F_{A} = 0.35, [η] = 1250 | No chitosan |
| 0.25 | 32 | 36 | 52 |
| 0.5 | 29 | 37 | 87 |
| 1 | 40 | 83 | 97 |
| 2 | 71 | 97 | 98 |
| 19 | 99 | 100 | 99 |

### EXAMPLE 6

Release of Ibuprofen from Chitosan

Ibuprofen (100 mg) and chitosan (various degrees of acetylation) (250 mg) were added to a diluted aqueous HCl solution at pH 2 (10 ml). The mixture was stirred for 30 minutes at 80° C., cooled to room temperature, transferred to a dialysis tube (cut off 12-14 kDa) and dialysed against tris buffer pH 7 (100 ml). The amount of ibuprofen in the dialysate was determined by UV.

The amounts of ibuprofen in dialysate are shown in Table 2 as a percentage of maximum detected amounts

TABLE 2

| Time for | | | Chitosan |
|---|---|---|---|
| dialysis | Chitosan | Chitosan | F_{A} = 0.35, |
| (hours) | F_{A} = 0.19, [η] = 610 | F_{A} = 0.46, [η] = 1230 | [η] = 1250 |
| 0.25 | 24 | 16 | 3 |
| 0.5 | 33 | 10 | 7 |
| 1 | 61 | 13 | 9 |
| 1.5 | 66 | 14 | 11 |
| 2 | 85 | 24 | 18 |
| 3 | 100 | 22 | 21 |

### EXAMPLE 7

Preparation of Warfarin/Chitosan Salt

A suspension of chitosan (0.50 g, F_{A}=0.40) in 0.1 M acetic acid (20 ml) in water was heated at reflux for 30 mins until the chitosan was dissolved. The acidic mixture was neutralized with 1 M NaOH. Warfarin (0.38 g, 1.2 mmol) was added and the mixture continuously stirred at reflux for an additional 1 h. The reaction mixture was evaporated in vacuo and finally freeze dried to yield the salt as a white powder (1.09 g).

### EXAMPLE 8

Preparation of Amoxycillin/Chitosan Salt

A suspension of chitosan (0.50 g, F_{A}=0.40) in 0.1 M acetic acid (20 ml) in water was heated at reflux for 30 mins until the chitosan was dissolved. The acidic mixture was neutralized with 1 M NaOH. Amoxycillin (0.52 g, 1.2 mmol) was added and the mixture continuously stirred at reflux for an additional 1 h. The reaction mixture was evaporated in vacuo and finally freeze dried to yield the salt as a green/yellow powder (1.17 g).

### EXAMPLE 9

Preparation of Amphotericin B/Chitosan Salt

A suspension of chitosan (0.50 g, F_{A}=0.40) in 0.1 M acetic acid (20 ml) in water was heated at reflux for ½ hour until the chitosan was dissolved. The acidic mixture was neutralized with 1 M NaOH. Amphotericin B (0.75 g, 0.80 mmol) was added and the mixture continuously stirred at reflux for an additional 1 h. The reaction mixture was evaporated in vacuo and finally freeze dried to yield the salt as a yellow powder (1.42 g).

### EXAMPLE 10

Release of Warfarin from Warfarin/Chitosan

The salt of warfarin/chitosan (from Example 7 above) (1.09 g) was suspended in a buffered solution with pH 7.4 (10 ml). The suspension was transferred into the dialysis tube (cut off 12-14 kDa) before the tube was transferred into a buffered solution of pH 7.4 (100 ml) under continuous stirring. 2 ml samples of the dialysate were taken at different times and the UV-absorbances measured with an UV-apparatus at 293 nm. As a control experiment, warfarin (0.38 g, 1.2 mmol) was dissolved in a buffered solution of pH 7.4 (10 ml) and transferred into the dialysis tube (cut off 12-14 kDa). 2 ml samples of the dialysate were taken at different times and the UV-absorbances measured with an UV-apparatus at 293 nm. The amounts of warfarin in dialysate are shown in Table 3 as a percentage of maximum detected amounts.

TABLE 3

| Time (hours) | Chitosan/warfarin | Warfarin |
|---|---|---|
| ½ | 4.7 | 14.8 |
| 2½ | 43.7 | 49.8 |
| 4 | 48.8 | 49.0 |
| 20 | 90.7 | 100 |

### EXAMPLE 11

Preparation of Pravastatin/Chitosan Salt

Pravastatin tablets (Bristol-Myers Squibb) (40 tablets each containing 20 mg pravastatin sodium) were crushed using a morter and pestle and the powder mixture added to 50 mL water. The mixture was added dropwise to 1 M HCl at pH 2 and the mixture extracted with chloroform (3×75 mL). The combined organic phase was dried (MgSO₄), filtered and evaporated in vacuo to yield pravastatin as a white powder (0.72 g).

A suspension of chitosan (0.50 g, F_{A} 0.40) in 0.1 M acetic acid (20 mL) was heated to reflux for 0.5 h until the chitosan was dissolved. The acidic mixture was neutralized with 1 M NaOH. Pravastatin (0.53 g, 1.2 mmol) was added and the mixture was continuously stirred at reflux for an additional 1 h. The reaction mixture was evaporated in vacuo and finally freeze dried to yield the salt as a brown powder (1.10 g)

### EXAMPLE 12

Effect of Chitosan on Availability of Norfloxacin

Norfloxacin (100 mg) and chitosan (F_{A}=0.35, η=1250) (250 mg) were added to a diluted aqueous HCl solution pH 2 (10 ml). The mixture was stirred for 2 hours at 80° C., cooled to room temperature and dialysed against tris buffer pH 7 (100 ml). The amount of norfloxacin in dialysate was determined by UV.

An experiment without chitosan was performed as a comparison.

The amounts of norfloxacin in dialysate are shown as a percentage of maximum detected amounts. The results are shown in Table 4.

TABLE 4

| Time for dialysis | | |
|---|---|---|
| (hours) | Without chitosan | With chitosan |
| 0.25 | 66 | 48 |
| 0.5 | 72 | 72 |
| 1 | 100 | 93 |
| 2 | 100 | 100 |
| 4 | 100 | 100 |

1. A pharmaceutical composition comprising a, physiologically active agent and a release sustaining or mucoadhesive agent, characterized in that said release sustaining or mucoadhesive agent comprises a chitosan having a F_{A} of between 0.40 and 0.80.

2. A pharmaceutical composition comprising a physiologically active agent and a release sustaining or mucoadhesive agent, characterized in that said release sustaining or mucoadhesive agent comprises at least two chitosans having different F_{A} values.

3. The composition as claimed in claim 2, wherein the F_{A} values of said chitosan differ by at least 0.2.

4. The composition as claimed in claims 1 or 3, wherein one or more of said chitosans has an F_{A} value below 0.40.

5. The composition as claimed in claims 1 or 3, wherein one or more of said chitosans has an F_{A} value below 0.25.

6. The composition as claimed in claims 2 or 3 comprising a chitosan having a F_{A} of from 0.25 to 0.80.

7. A composition as claimed in claims 2 or 3 comprising a chitosan having a F_{A} of between 0.40 and 0.80.

8. The composition as claimed in claim 1 comprising a chitosan having an F_{A} between 0.40 and 0.60.

9. The composition as claimed in claim 8 comprising a chitosan having a F_{A} between 0.40 and 0.55.

10. The composition as claimed in claim 1, wherein said release sustaining or mucoadhesive agent is present in a solid or liquid crystalline micro- or nano-structure.

11. The composition as claimed in claim 10, wherein said release sustaining or mucoadhesive agent is present in a nanoparticle, a liposome, a micelle, a reversed micelle or a fragmented cubic or hexagonal phase liquid crystal.

12. The composition as claimed in claim 1, wherein said physiologically active agent is a compound with a molecular weight of up to 500 g/mol.

13. The composition as claimed in any claim 1, wherein said physiologically active agent is a protein or a peptide with a molecular weight of up to 7,000 g/mol.

14. The composition as claimed in claim 1, wherein said physiologically active agent is selected from the group consisting of analgesics, anti-inflammatories, hormones, antiparasitics, antineoplastics, antihypertensives, anti-ulcer drugs, antidepressants and cholesterol reducing agents.

15. The composition as claimed in claim 1, wherein said physiologically active agent is an acidic water-soluble drug.

16. The composition as claimed in claim 15, wherein said physiologically active agent is selected from the group consisting of acetylsalicylic acid, ibuprofen, antibiotics and anticoagulants.

17. The composition as claimed in claim 1 containing a chitosan fully water-soluble at a pH of 3 to 7.

18. The composition as claimed in claim 17, wherein said chitosan is fully water-soluble at a pH of from 6 to 7.

19. The composition as claimed in claim 1 containing chitosans having a weight average molecular of from 1,000 to 5,000,000 g/mol.

20. The composition as claimed in claim 19 containing chitosans having a weight average molecular weight of from 10,000 to 30,000,000 g/mol.

21. The composition as claimed in claim 1 containing from 20 to 90% by weight of chitosan.

22. The composition as claimed in claim 1 containing chitosan and said physiologically active agent in a weight ratio in the range 20:1 to 0.5:1.

23. A pharmaceutical composition comprising admixed at the molecular level a solid mixture of a chitosan and a physiologically active agent.

24. The composition as claimed in claim 23 comprising a chitosan having a F_{A} of between 0.40 and 0.80.
